(19) United States
(12) Patent Application Publication
(10) Pub. No.: US 2017/0295758 A1
(43) Pub. Date: Oct. 19, 2017

(54) DEVICE AND METHOD FOR KEEPING ANTS OUT OF LIQUID FOOD

(71) Applicant: Kathryn Graves, Croton-on-Hudson, NY (US)

(72) Inventor: Kathryn Graves, Croton-on-Hudson, NY (US)

(21) Appl. No.: 15/130,661

(22) Filed: Apr. 15, 2016

(51) Int. Cl. A01K 39/01; A01K 67/033; A01K 39/04; F16M 13/00; A01K 39/02

(52) U.S. Cl. CPC A01K 39/0113; F16M 13/00; A01K 39/0206; A01K 39/04; A01K 67/033

(57) ABSTRACT

A device keeps ants out of a feeder for flying creatures, such as hummingbirds. The feeder is adapted to be held above the ground by a supporting member. The device may include an annular vessel, fillable by a fluid, such as water. The annular vessel can surround the supporting member, below the feeder, for instance on the ground. The feeder may thus be disposed to be visible from a window or above a wall, while the annular vessel is below a sill of the window or the top of the wall. The annular vessel is designed to be passed over the supporting member, when the feeder is not attached.

### FIELD

A device keeps ants out of liquid food in an outdoor feeder for flying creatures.

### BACKGROUND

Ants are attracted to sweetened liquid food. Such liquid food may include water and dissolved sugar. This type of food can be used for flying wild creatures such as hummingbirds, orioles, and butterflies.

An existing hummingbird feeder available through backyardwildbirds.com features a guard containing water to keep ants out of the feeder. This feeder is suspended from above. In this position, the guard is disposed immediately above the feeder, and about a vertical axis that passes through the feeder.

Users commonly select feeders based on appearance, to enhance the beauty of their homes.

Users of hummingbird feeders will commonly hang such feeders outside a window, so that the users can watch the hummingbirds feeding. When the above-mentioned feeder/guard assembly is hung outside a window, the proximity of the guard to the feeder makes the guard visible to the user. This is unaesthetic.

### SUMMARY

It is desirable to convert an existing aesthetic feeder into one that also resists ants.

Where a feeder is supported by a post, a barrier may be added about the post, below the feeder. When the feeder is displayed outside a window or behind a wall, the feeder will be above a sill of the window, or the top of the wall, but the barrier will be below the sill or top.

### BRIEF DESCRIPTION OF THE DRAWING

Embodiments will now be described, by way of non-limiting example, with reference to the following figures:

FIG. 1 shows a vertical section during a process for putting an ant barrier device over a conventional hummingbird feeder

FIG. 2 shows a top view of the hummingbird feeder with the device installed.

FIG. 3 shows the device installed on the hummingbird feeder and hidden behind a low wall.

### DETAILED DESCRIPTION

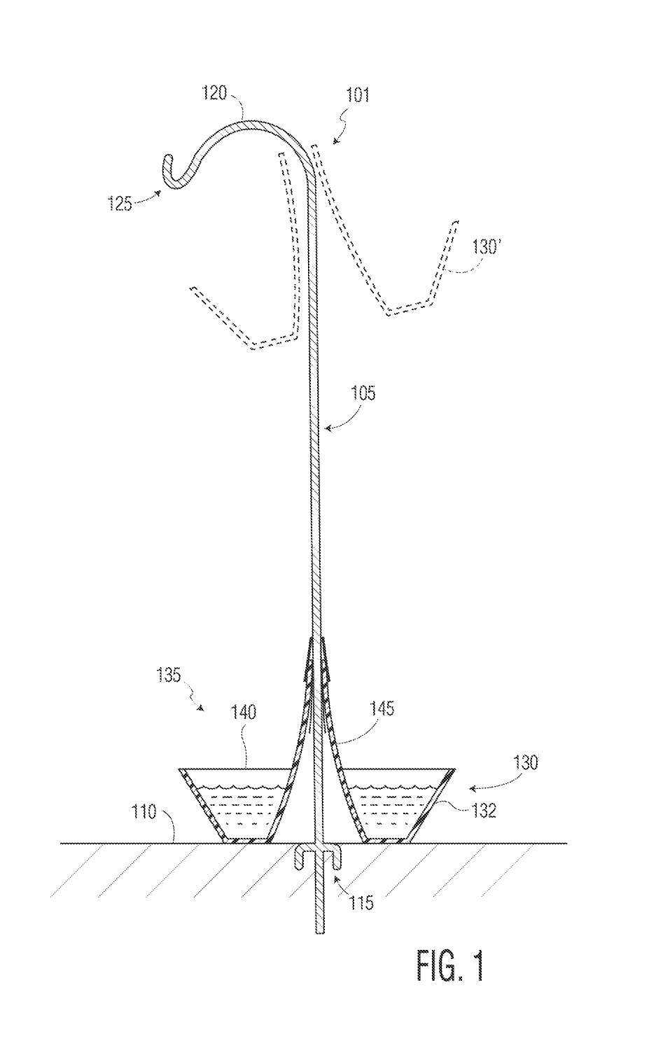

FIG. 1 shows a support member 101 for a conventional outdoor hummingbird feeder. The member includes a post 105, anchored in the ground 110, by means of an anchoring device 115. The support member also includes a rounded top 120 and a hook 125 for holding the feeder.

With the feeder (not shown) off, an ant barrier 130′ can be slid over the hook 125, rounded top 120, and post 105 to rest on the ground 110 at position 130.

The barrier 130, 130′ includes a dish portion 132, which forms an annular cavity 135. The cavity is fillable with a fluid barrier 140. The fluid barrier 140 will typically be water. Ants will not cross the water.

The barrier 130, once in place, may be sealed to the post 105 to hinder ants from crawling underneath and up the post. Sealing may be effectuated by a conical sleeve 145 coupling the annular cavity 135 to the post 105. The conical sleeve may be a flexible material, such as plastic. The conical sleeve 145 may be sealed to or integral with the dish portion 132.

Alternatively, the dish portion 132 may not have a conical sleeve. It may be simply an annular container for fluid. It may be slipped over the post. Then a flexible adhesive sheet material, such as duct tape, may be used to hinder ants from coming under the container and up through the center.

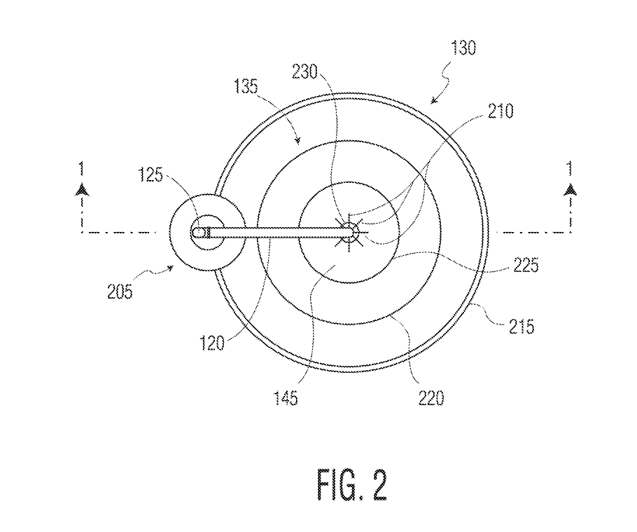

FIG. 2 shows a top view of an assembly including the ant barrier 130, the rounded top 120 of the support member, and the feeder 205 on the hook 125. The annular cavity 135 surrounds the post 105, which is not visible in this view. Conical sleeve 145 includes slits 210 in the top end to allow the sleeve and the barrier to pass over the hook 125 and the rounded top 120 of the support member 101.

Visible are the outer edge of the device 215, the outer edge 220 of the bottom of the annular cavity, the inner edge 225 of the bottom of the annular cavity, and the edge 230 of the top of the conical sleeve.

The top end of the conical sleeve 145 may alternatively be a flexible ring that stretches to fit over support members of differing thicknesses.

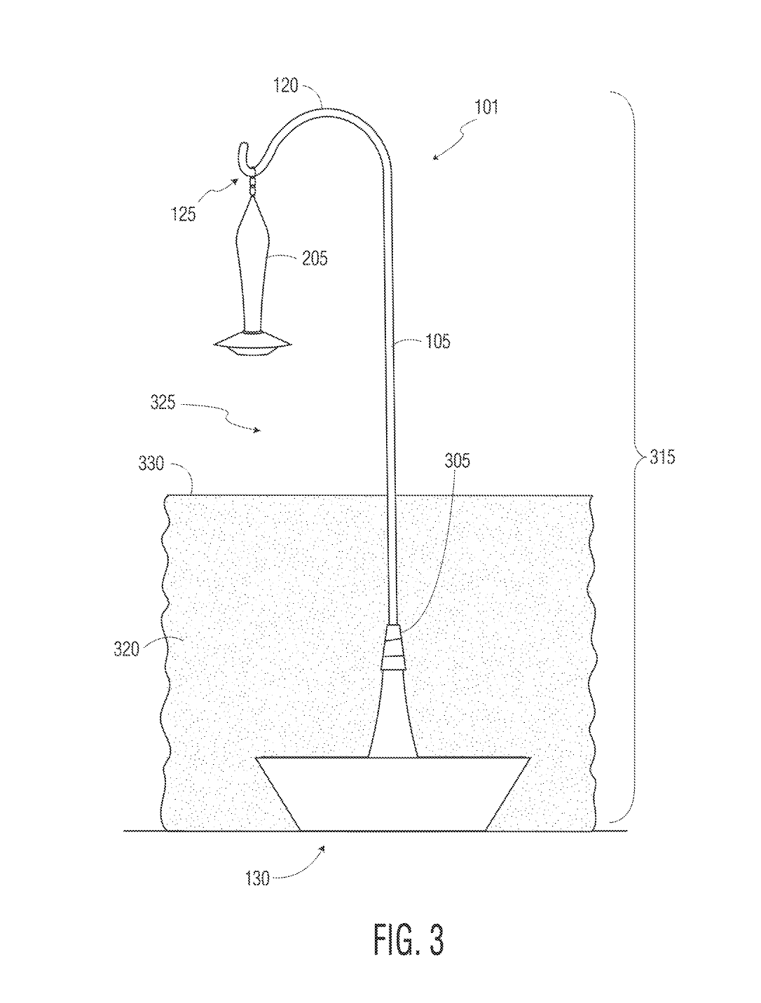

FIG. 3 shows a side view of a barrier device 130 in place. Sealing tape 305 has been placed over the slits to further hinder ants from coming under the barrier. The feeder 205 has been hung from the hook 125. The assembly 315 has been positioned behind a wall 320, such as a garden wall or a wall ringing a patio. The feeder 205 is in the space 325 above the wall.

Alternatively, the wall 320 may be part of a building, such as a house, and the space 325 may be a window of the house. In any case the barrier device 130 is below a top 330 of the wall 320. This top 330, may also be a window sill.

From reading the present disclosure, other modifications will be apparent to persons skilled in the art. Such modifications may involve other features that are already known in the design, manufacture and use of bird feeders, and which may be used instead of or in addition to features already described herein. Although claims have been formulated in this application to particular combinations of features, it should be understood that the scope of the disclosure of the present application also includes any novel feature or novel combination of features disclosed herein either explicitly or implicitly or any generalization thereof, whether or not it mitigates any or all of the same technical problems as does the present invention. Applicant hereby gives notice that new claims may be formulated to such features during the prosecution of the present application or any further application derived therefrom.

The word “comprising”, “comprise”, or “comprises” as used herein should not be viewed as excluding additional elements. The singular article “a” or “an” as used herein should not be viewed as excluding a plurality of elements. Unless the word “or” is expressly limited to mean only a single item exclusive from other items in reference to a list of at least two items, then the use of “or” in such a list is to be interpreted as including (a) any single item in the list, (b) all of the items in the list, or (c) any combination of the items in the list. Use of ordinal numbers, such as “first” or “second,” is for distinguishing otherwise identical terminology, and is not intended to imply that operations or steps must occur in any particular order, unless otherwise indicated.

1. A device for keeping ants out of an outdoor liquid food feeder, the feeder being adapted to couple with a support member, the device comprising a barrier container adapted to couple with and surround the support member at a position below the feeder and to hold a barrier fluid around the support member, so that the barrier fluid is impassible for ants.

2. The device of claim 1, wherein the feeder is a hummingbird feeder.

3. The device of claim 1, wherein the feeder contains a sugar solution.

5. The device of claim 1, wherein the feeder is a butterfly feeder.

6. The device of claim 1, wherein the feeder is an oriole feeder,

7. The device of claim 1, wherein the barrier container forms an annular shape.

8. The device of claim 1, wherein the barrier fluid comprises water.

9. The device of claim 1,
wherein the support member comprises a post; and
the device further comprises
an annular cavity adapted to hold the barrier fluid in the form of a moat about the post; and
a coupling sleeve adapted to slide along the post to position the device with respect to the post.

10. The device of claim 9, wherein:
the support member comprises a curved top and a bent end for coupling with the food container; and
the coupling sleeve forms slits for opening up while the device is sliding past the bent end and curved top.

11. The device of claim 10, further comprising sealing means for closing the slits once the device is in place.

12. The device of claim 9, wherein the coupling sleeve comprises
a first, larger end for coupling with the annular cavity,
a second, smaller end for coupling with the post;
a flexible, hollow body coupled between the first and second ends, adapted to surround the post and seal a space between the annular cavity and the post, to hinder ants from coming from underneath.

13. A method for modifying an outdoor feeder to resist ants, the method comprising:
placing a post support member of the feeder in the ground, without the feeder attached;
sliding a barrier device over the post support member, until the barrier device rests on the ground; and
attaching the feeder to the post support member.

14. The method of claim 13, wherein the barrier device comprises an annular cavity for holding a barrier fluid and a flexible sleeve for coupling with the post, the method further comprising
sealing the sleeve to the post, so that ants cannot come in from underneath; and
filling the annular cavity with water.

15. A device for keeping ants out of liquid food stored outdoors for wild, flying creatures, the device comprising:
annular cavity means for holding a liquid barrier fluid so that the fluid forms a moat around a supporting apparatus that is for supporting the food, the annular cavity means being positioned so that the moat is below the liquid food; and
means for sealably coupling the annular cavity means with the supporting apparatus, so that ants are hindered from coming from below.

16. The device of claim 15, the device is for sitting on the ground, while the food is above the ground, so that when the food is viewed from a window or over a wall, the device is below a sill of the window or the top of the wall.
